(19) United States
(12) Patent Application Publication
(10) Pub. No.: US 2022/0125073 A1
(43) Pub. Date: Apr. 28, 2022

(54) TREATMENT AND CONTROL OF COLONY COLLAPSE DISORDER

(71) Applicant: Tallgrass Biologics L.L.C., Worthington, MN (US)

(72) Inventor: Ben Hause, Slayton, MN (US)

(21) Appl. No.: 17/277,771

(22) PCT Filed: Sep. 20, 2019
(86) PCT No.: PCT/US2019/052096

§ 371 Date: Mar. 19, 2021

Related U.S. Application Data
(60) Provisional application No. 62/733,822, filed on Sep. 20, 2018.

(51) Int. Cl. A23K 20/147; A23K 10/18; A23K 10/20; A23K 10/30; A23K 20/111; A23K 20/163; A23K 20/158; A23K 20/174; A23K 20/189; A23K 20/195; A23K 40/10; A23K 40/20

(52) U.S. Cl. CPC A23K 20/147; A23K 10/18; A23K 10/20; A23K 10/30; A23K 20/111; A23K 20/163; A23K 50/90; A23K 20/174; A23K 20/189; A23K 20/195; A23K 40/10; A23K 40/20; A23K 20/158

(57) ABSTRACT

A prophylactic and/or therapeutic food composition for control of deformed wing vims (DWV) in bees and/or bee larvae, and methods of using and making the same. Bee feed compositions comprising anti-DWV antibodies dispersed in an edible base composition. Use of recombinant VP1, VP2, VP3, and/or VP4 capsid proteins, or VLPs of deformed wing vims (DWV) to passively generate anti-DWV antibodies in chicken egg yolks, and use of such anti-DWV antibodies for prophylaxis or treatment of deformed wing virus in the bees and/or larvae.

### CROSS-REFERENCE TO RELATED APPLICATIONS

The present application claims the priority benefit of U.S. Provisional Patent Application Ser. No. 62/733,822, filed Sep. 20, 2018, entitled TREATMENT AND CONTROL OF COLONY COLLAPSE DISORDER, incorporated by reference in its entirety herein.

### SEQUENCE LISTING

The following application contains a sequence listing in computer readable format (CRF), submitted as a text file in ASCII format entitled “Sequence_Listing,” created on Sep. 18, 2019, as 14 KB. The content of the CRF is hereby incorporated by reference herein.

### BACKGROUND OF THE INVENTION

### Field of the Invention

The present invention relates to the use of antibodies against Deformed Wing Virus for prophylaxis and/or treatment of colony collapse disorder in honeybees.

### Description of Related Art

Worldwide, honeybee (Apis mellifera) colonies have seen dramatic population losses, a phenomenon called colony collapse disorder (CCD). Numerous causative factors have been investigated, including loss of habitat and plant diversity, increasing use of insecticides and several bee pathogens, including bacteria, fungi, mites, and viruses. The spread of CCD across the globe has correlated with the introduction of the bee parasitic mite Varroa destructor. For example, the introduction of Varroa destructor on the Hawaiian islands in the early 2000's coincided with reports of CCD. While the Varroa mites feed on both pupa and adult bees, they have been eliminated as a direct cause of CCD as mite control measures utilized in collapsing colonies minimize mite colonization, yet fail to prevent CCD.

Deformed wing virus (DWV) is a member of the genus Ilfavirus, family Picornavirales. DWV replicates in a number of insect hosts, including both Varroa and Apis. In bees, genetically diverse DWV have been classified as one of three master variants, types A, B and C (Kevill et al., Method development and application to quantify the role three DWV master variants in overwinter colony losses of European honeybees. Viruses 9:314, 2017). A complex mix of DWV quasi-species are often found within a colony. While occasional overt signs of disease caused by DWV have been observed, (mainly adults with deformed wings), DWV and Apis have long co-existed without widespread bee losses.

Varroa can transmit DWV to bees through mite feeding behavior. Bees infected with DWV via mite feeding have been shown to have extremely high levels of DWV. Importantly, a concurrent loss in DWV genetic diversity is observed. The results suggest that DWV replication in Varroa has selected for DWV with increased virulence in bees. In addition, direct injection of DWV into bees by mite feeding leads to significantly increased viral titers as compared to bee infection via oral feeding.

Current CCD prophylaxis and treatment focus on control of Varroa. While these efforts are often helpful, increased efficacy is necessary. Unlike vertebrates which have both innate and adaptive immunity, insects possess only innate immunity. The insect immune system can recognize conserved pathogen features via pattern recognition receptors which activates general anti-pathogen defenses. The insect genomes however lack the ability to mount a specific, pathogen-specific immune response. In vertebrates, activation of the adaptive immune system employs somatic cell recombination in B and T lymphocytes which gives rise to highly specific antibody and cell-based responses to the pathogen. Insects lack this ability.

Vertebrates are often born with an immune system that is not fully mature and consequently lack the ability to generate adaptive immune responses. To overcome this deficiency, maternal antibodies are delivered to the newborn to provide passive protection while the immune system matures. In avian species, maternal antibodies can be found in the egg yolk.

In mammals, maternal antibodies are passed to the newborn via colostrum and nursing. These antibodies are referred to as passive as they are formed by the mother and transferred to the newborn. In mammals, these antibodies typically have a half-life of approximately two weeks and often provide protection from the particular pathogen for the first month or two of life. Passive antibodies have been exploited for the protection of animal health. For example, vaccination of pregnant females is utilized to generate an immune response which will be transferred to neonates via nursing. In biotechnology, animals are often hyperimmunized with a pathogen antigen to generate high antibody titers. Antibodies are subsequently purified from the hyperimmunized animal sera and injected into susceptible or infected animals. Likewise, antibodies are purified from hyperimmunized animal colostrum or eggs and used to feed susceptible or infected neonates to provide protection. Recently, researchers in China hyperimmunized chickens using whole inactivated Sacbrood virus and isolated antibodies from egg yolk (Sun et al., Preparation and application of egg yolk antibodies against Chinese Sacbrood virus infection. Frontiers Microbiology 9:1814, 2018). Bees feed anti-Sacbrood virus antibodies were protected from disease caused by Sacbrood virus. The use of antibodies in bees is unexpected, and it is unknown whether similar approaches would work for other pathogens.

### SUMMARY OF THE INVENTION

The present invention is broadly concerned with the passive generation of antibodies against DWV in chickens (“chicken- or egg-derived antibodies”) and their use for antibody prophylaxis and therapeutics against CCD in honeybee colonies. In one exemplary embodiment, one or more recombinant antigens of DWV are expressed by a recombinant baculovirus vector and used to immunize chickens. The method can include one or more antigens of DWV expressed in insect cells. In one or more embodiments, the invention is concerned with prophylactic and/or therapeutic bee (or larvae) feed or supplements comprising anti-DWV antibodies, and preferably anti-DWV-A antibodies, dispersed in an edible base composition. The edible base composition typically comprises a carbohydrate source or is formulated for mixing with a carbohydrate source. The anti-DWV antibodies can be used in liquid solution or as lyophilized dry form with commercial bee feed or feed supplements, such as traditional sugar syrup (sucrose), patties, granules, powders, compressed tablets, and the like, such as Mann Lake Ultra Bee Dry Feed. Additional beneficial ingredients, such as amino acids, vitamins (e.g., B complex), fats/lipids, probiotics (e.g., L. acidophilus, E. faecium, B. bifidum, B. licheniformis, B. pumilus, and/or L. plantarum), enzymes/prebiotics, essential oils, yeast, plant polyphenols, phytonutrients, minerals, herbs, proteins, and carbohydrates can be included in the feed or supplement.

Commercial supplements or feed substitutes are available, such as Purina® Hearty Bee™, SuperDFM-HoneyBee, MegaBee®, Nozevit Plus, and the like. Various supplements can be used dry, or mixed with sugar syrup. Supplements and/or feed are also formulated as compressed tablets or patties (e.g., Bee-Pro Patties+, Ultra Bee Patties, etc.), and fed to the bees and/or larvae. Such feed may be used as a supplement or substitute for the honeybees' natural diet, and offered inside the hive or brood box, or freely outside in the vicinity of the hive where bees will otherwise come into contact with the feed. Additional base ingredients used in such feed substitutes often include vegetable oils, such as canola or sunflower oil, and grain flours, such as soy, sorghum, or wheat flour, corn meal, and yeast, such as brewer's yeast. A carbohydrate/sugar source is added to the feed in the form of sucrose, corn syrup, sugar syrup, cane, or beet sugar.

Thus, embodiments of the invention further comprise methods of prophylaxis or treatment of deformed wing virus in a bee colony. The anti-DWV antibody composition can be used in conjunction with additional colony treatments for disease and/pests, such as a those containing antibiotics, miticides, formic acid, nutraceuticals (thymol), etc. for honeybee dysentery, mites, parasites, etc. Preferably the anti-DWV antibodies have been purified from chicken eggs, more preferably from hens immunized with recombinant VP1, VP2, VP3, and/or VP4 capsid proteins, or recombinant VLPs. Regardless, the anti-DWV antibody composition is fed to and ingested by the bees and/or larvae at therapeutically-effective dosages and/or timing intervals to prevent and/or treat DWV in the bee colony. As used herein, the term “therapeutically effective” refers to the amount and/or time period that will elicit the biological response in the treated insect or larvae being sought by a researcher or clinician, and in particular elicit protection against DWV symptoms and according reduce mortality. For example, in one or more embodiments, therapeutically effective amounts and time periods are those that deliver an effective amount of anti-DWV antibodies to the bees and/or larvae being treated. One of skill in the art recognizes that an amount or time period may be considered “therapeutically effective” even if the condition is not totally prevented or eradicated but improved partially. Preferably, methods of the invention are effective in reducing incidence of mortality due to DWV by at least 50% in a beehive or colony (50% or less of bees exhibiting signs of DWV), and even more preferably by at least 60%.

### BRIEF DESCRIPTION OF THE DRAWINGS

FIG. 1 shows the ELISA results for processed DWV antibody samples.

FIG. 2 is a graph of the viral titer in larval tissue from the different experimental groups;

FIG. 3 is a graph of the cumulative mortality (%) in the larvae from the different experimental groups at day 5.

### DETAILED DESCRIPTION

The following description set forth methods in accordance with the invention. It is to be understood, however, that these examples are provided by way of illustration and nothing therein should be taken as a limitation upon the overall scope of the invention.

### Generation of DWV Antibodies

DWV consists of three master variant genotypes (A, B and C) which include a wide array of genetic diversity (Kevill et al., 2017). Both types A and B replicate within mites and bees and frequently recombine. Bees collected from colonies experiencing CCD typically contain master variant A DWV which is consequently considered highly virulent. In contrast, colonies infected with DWV variant B are often protected from CCD (Mordecai et al., Diversity in a honeybee pathogen: first report of a third master variant of the deformed wing virus quasi-species. ISME Journal 10:1264-1273, 2016).

In one or more embodiments, antibodies against DWV variant type A are generated by hyperimmunization of chickens with DWV antigen. Whole virus DWV can be isolated from infected bees displaying symptoms of deformed wings and confirmed by molecular means such as real time reverse transcription PCR as previously described (Kevill et al., 2017). DWV is chemically inactivated with formalin or binary ethyleneimine and mixed with an oil-in-water type adjuvant. Alternatively, and preferably, one or more capsid proteins of DWV is instead produced recombinantly using a suitable expression system. The non-enveloped virion of DWV is composed of four capsid proteins, VP1 (SEQ ID NO:1), VP2 (SEQ ID NO:2), VP3 (SEQ ID NO:3), and VP4 (SEQ ID NO:4).

Baculoviruses are viruses which infect insects. Baculoviruses have been exploited by biotechnology for the high-level production of recombinant proteins owing to their properties of ease of genetic engineering, ability to grow in economical, defined, animal-component-free media, and ability to produce large amounts of protein in vitro in suspension or attached cells. Also, owing to their tropism limited to insect cells, baculoviruses exhibit a broad safety profile. Autographa californica nuclear polyhedrosis virus (AcNPV) is one of the most extensively studied baculoviruses, and has become the prototype baculovirus. Baculovirus expression systems and baculovirus expression vectors in general have been described extensively in the literature, including U.S. Pat. No. 4,745,051, O'Reilly at al. (Baculovirus Expression Vectors, A Laboratory Manual. (1993)), and Murhammer (Baculovirus and Insect Cell Expression Protocols. In: Methods in Molecular Biology™. Volume 388 (2007)). A variety of commercial baculovirus-based expression systems are also available, including BaculoGold™ DNA (PharMingen), Bac-N-Blue™ DNA (Invitrogen), or BacPAK6™ DNA (Clontech) for co-transfection with the transfer vector (donor or shuttle) plasmid DNA containing the foreign gene. Alternatively, insect cells are transfected with a recombinant bacmid DNA constructed by transposition of the donor plasmid DNA in E. coli cells, using the Bac-to-Bac™ (Invitrogen-Gibco/Life Technologies) system.

In one or more embodiments, VP1, VP2, VP3, and/or VP4 genes (codon-optimized for expression in insect cells) are chemically synthesized and cloned into a plasmid in E. coli such that the genes are operably linked to an insect cell promoter and terminator. In addition, a baculovirus secretion signal sequence (e.g., GP67) and affinity tag (e.g., polyhistidine-tag) are often added to the gene during synthesis to facilitate secretion of the protein into the supernatant during cell culture and purification of the protein by affinity chromatography, respectively. Isolation and purification from culture medium is considerably easier than purification from cell lysates, as cellular material does not need to be removed from the preparation. The transfer plasmid is recombined with baculovirus genomic DNA in insect cells to generate recombinant baculovirus that can express VP1, VP2, VP3 and/or VP4. The recombinant baculoviruses are propagated in suitable insect cells and the recombinant capsid protein is secreted into the media and purified by affinity chromatography. Exemplary insect cells are those derived from the Lepidopteran species Spodoptera frugiperda, Trichplusia ni, Bombyx mori, cell lines derived therefrom, and the like. Particularly preferred insect cell lines include SF9 (and variants), SF21, High Five (BTI-TN-5B1-4), and the like. Culture media suitable for insect cell culture is preferably serum-free, and various formulations are known in the art and widely available, such as SF90011.

VP1, VP2, VP3, and/or VP4 can be produced individually by infection of insect cells with the recombinant baculovirus strain engineered to express each protein. Alternately, insect cells can be co-infected with multiple recombinant baculoviruses to concurrently express combinations of VP1, VP2, VP3, and/or VP4. Co-expression of capsid proteins in baculovirus leads to formation of virus like particles (VLPs) which are morphologically and antigenically indistinguishable from native virions however lack encapsidated genetic material. VLPs have been widely used as vaccine antigens owing to their antigenic properties and safety as they are non-infectious.

It will be appreciated that expression of recombinant VP1, VP2, VP3, and/or VP4 capsid proteins, or VLPs is not necessarily limited to a baculoviral expression system. The recombinant proteins can also be expressed in other expression vectors such as yeast, mammalian, or bacterial expressions systems.

Regardless of the embodiment, the purified, inactivated whole DWV or purified recombinant VP1, VP2, VP3, VP4, or VLPs (composed of combinations of VP1, 2, 3, or 4) are combined with a pharmaceutically-acceptable carrier to create the immunizing formulation and used to immunize laying hens. In preferred embodiments, recombinant antigens are used in the immunizing formulation to immunize the hens. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the administered antigens as would be well known to one of skill in the art. In one or more embodiments, the purified, inactivated whole DWV or purified recombinant VP1, VP2, VP3, VP4, or VLPs are combined with an oil-in-water adjuvant such as Freund's incomplete adjuvant at 50% or Emulsigen (MVP laboratories) at 20% final volume to create the immunizing formulation. In one or more embodiments, laying hens are injected with the immunizing formulation biweekly at least twice to initiate a strong initial immune response, and thereafter periodically boosted (e.g., one, two or more times) to maintain the immune response in the hens, resulting in passive generation of anti-DWV antibodies in eggs from the immunized hens.

### Antibody Purification and Quantification

Immunoglobulin Y is purified from egg yolk as previously described (Sun et al., 2018). Antibody titer is determined qualitatively by enzyme linked immunosorbent assay using purified VP1, VP2, VP3 and/or VP4 as antigen coated to the Immulon 2HB polystyrene 96 well plates at 1 ug/well. The purified antibodies as serially diluted two-fold down the 96-well plate and the titer is determined by the reciprocal dilution which yields an absorbance significantly greater than control non-immunized egg yolk antibodies using a secondary horseradish peroxidase (HRP) labeled anti-chicken antibody coupled to a suitable chemical substrate for HRP.

### Commercial Use

A commercial antibody solution is formulated such that it contains a protective titer of anti-DWV antibodies, and preferably anti-DWV-A antibodies. Bees are routinely fed a commercial sugar solution or supplements at multiple times per year at times of stress such as prior to winter and emergence in spring. The commercial anti-DWV antibody solution is composed of water, glucose, anti-DWV-A antibodies and preservatives. Alternately, the anti-DWV-A antibodies are lyophilized and sold as a solid composition which is suitably rehydrated with commercially-available bee feed, or use with dry bee feed or supplements.

The anti-DWV-A antibody composition is provided prophylactically prior to and post-winter hibernation. The anti-DWV-A antibody composition is also used therapeutically in response to signs of CCD, namely loss of bees in the colony or presence of individuals with deformed wings.

### Similar Products

Besides DWV, numerous other viruses cause losses to honeybee populations. Acute bee paralysis virus (ABPV), black queen cell virus (BQCV), Kashmir bee virus (KBV) and Israeli acute paralysis virus (IAPV) all cause honeybee mortality and prevention and control of disease caused by these viruses could be addressed by the same approach outlined above. Combinations of antibodies directed towards multiple viruses are expected to show increased efficacy given the multifactorial pathogenic nature of CCD.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### Examples

The following examples set forth methods in accordance with the invention. It is to be understood, however, that these examples are provided by way of illustration and nothing therein should be taken as a limitation upon the overall scope of the invention.

### Anti-DWV Antibodies

To produce anti-DWV antibodies, recombinant DWV surface capsid protein VP1, VP2 and VP3 peptides were expressed in a baculovirus expression system and used as antigens to immunize chickens. Recombinant baculoviruses that express VP1, VP2, and/or VP3 genes are constructed by way of homologous recombination between baculovirus DNA and chimeric plasmids containing the VP1, VP2, and/or VP3 genes sequences.

Specifically, codon-optimized VP1, VP2 and VP3 genes were each chemically synthesized and subcloned into a pFastBac1 expression vector using EcoR1, such that the genes are operably linked to an insect cell promoter and terminator, along with the GP67, SEQ ID NO:5 secretion signal, and 6× Histidine affinity tag. Resulting coding sequences are in SEQ ID NO: 6 (VP1), SEQ ID NO: 7 (VP2), and SEQ ID NO: 8 (VP3), each including the Kozack, stop codon, and HindIII sequences.

The transfer plasmid is recombined with baculovirus genomic DNA via transformation with DH10Bac E. coli strain to generate recombinant bacmid DNA which was subsequently used to transfect SF9 insect cells to generate recombinant baculovirus expressing VP1, VP2, and/or VP3. Transfection of SF9 cells was performed with Cellfectin II (ThermoFisher) per manufacturer's instructions. SF9 cells were grown in Sf-900 II SFM Expression Medium (Life Technologies). The cells were maintained in Erlenmeyer Flasks at 27° C. in an orbital shaker. One day before transfection, the cells were seeded at an appropriate density in 6 wells CORNING-COSTAR. On the day of transfection, DNA and Cellfectin II (Life Technologies) were mixed at an optimal ratio and then added into the plate with cells ready for transfection. Cells were incubated in Sf-900 IISFM for 5-7 days at 27° C. before harvest. The supernatant was collected after centrifugation and designated as P1 viral stock. P2 was amplified for later infection. The 1 L SF9 cell cultures were infected by P2 virus. Cells were incubated in Sf-900 II SFM for 3 days at 27° C. before harvest.

Cell pellets were harvested and lysed by cell lysis buffer containing 7 M guanidinium-HCl. The cell lysate supernatant was incubated with Ni Columns to capture the target protein. Higher purity fractions were pooled and followed by 0.22 μm filter sterilization. Proteins were analyzed by SDS-PAGE and Western blot by using standard protocols for molecular weight and purity measurements. The primary antibody for Western blot was Mouse-anti-His mAb (GenScript, Cat.No. A00186). The concentration was determined by Bradford protein assay with BSA as a standard.

The recombinant capsid proteins were then combined with Freund's incomplete adjuvant at 50% final volume and used to inject laying hens biweekly four times. Anti-DWV antibodies were subsequently purified from the egg yolks using published protocols and used for the study.

Briefly, twelve white leghorn chickens, approximately 6-months of age, were immunized four times with the recombinant DWV capsid protein vaccine. Each vaccine consisted of 25 μg/mL of affinity purified VP1, VP2 and VP3 and 50% incomplete Freund's adjuvant with the remaining volume comprised of phosphate buffered saline. The birds were administered with a 0.5 cc intramuscular injection in each breast. Immunizations were given at 14-day intervals. Eggs were collected daily, beginning two weeks after the third vaccination. Yolks were separated from the albumen using a wire egg separating apparatus. The yolks were then processed according to published protocols. Yolks were diluted with seven volumes of tap water and brought to a pH of 5.0 using 1.0 N HCl. This was done in 1-gallon containers, with each container consisting of 500 mL yolk, 3 L water, and 36 mL 1.0 N HCl. A pH probe was used to verify the pH of each aliquot. This solution was frozen overnight at −18° C. and thawed the following day. Upon thawing, the solution separated into two distinct layers: an upper aqueous layer (mostly transparent) containing the IgY and a lower viscous layer, containing lipids and other insoluble material (orange). The upper aqueous layer was collected with a pipet while the bottom lipid layer was discarded. The aqueous layer was then concentrated using MiniKros Hollow Fiber Filter Module; P/N: N04-E030-05-N; Media/Rating: mPES/30 k; Surface Area: 5400 cm²; Max.Op.Pressure 30 psig (2 bar); SN: 3303990-07/18-002. The volume of aqueous layer was reduced approximately 10-fold. It was estimated that each egg provides approximately 16.67 mL of yolk which when processed as above, yielded 11.05 mL of concentrated antibody.

As a secondary control antibody purification method, a commercial IgY purification kit was purchased from Exalpha (EggsPress IgY Purification Kit, cat. #lk2000). DWV antibodies were purified from egg yolks per manufacturer's instructions.

### Antibody Analysis

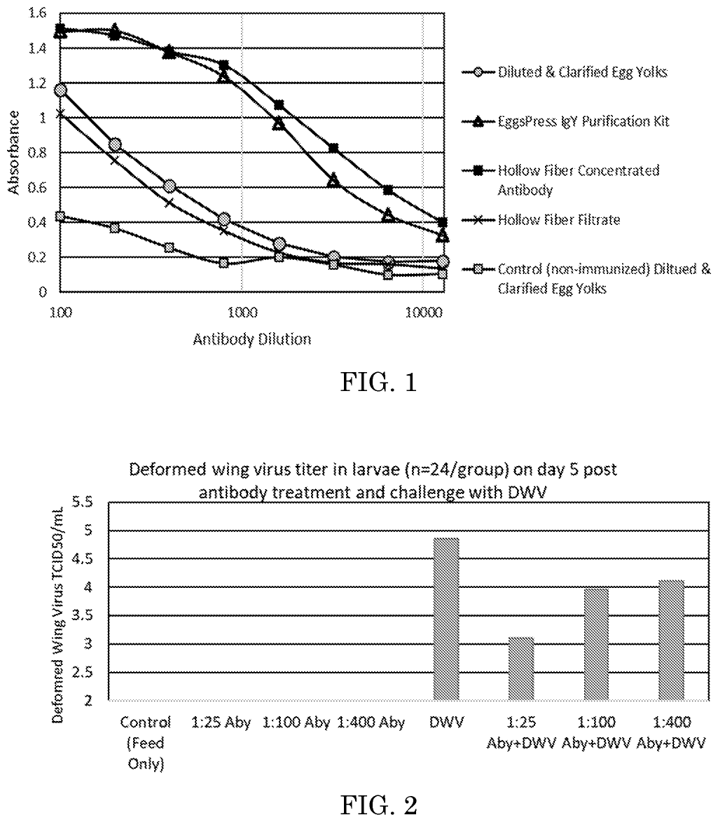

Fractions collected during the antibody purification process were analyzed for antibodies specific to DWV VP1, VP2 and VP3 by enzyme linked immunosorbent assay (ELISA). IgY was purified by either the water dilution/freeze-thaw method (Diluted and Clarified Egg Yolks) followed by hollow fiber concentration (Hollow Fiber Concentrated Antibody) or a commercial IgY purification kit (EggsPress IgY Purification Kit). Hollow Fiber Filtrate is material removed by the filter and egg yolks collected from non-immunized birds were included as negative controls. The results are in FIG. 1. Briefly, Immulon 2HB 96-well plates were coated with purified recombinant VP1, VP2 and VP3 by diluting the recombinant proteins to 1 μg/mL in carbonate-bicarbonate buffer (Sigma Aldrich C3041) and adding 100 μL to each well and incubating overnight at 4° C. Fluids were dumped from the plate and the wells were next blocked with 100 μL Superblock (ThermoFisher 37517) for 1 hour at 37° C. Next, 100 μL of Superblock were added to all wells. Samples were diluted in a 1.5 mL microcentrifuge tube 1:50 in Superblock, and 100 μL of each sample was then added to a well in row A of the plate and mixed with a pipette. Next, 100 μL from the well in row A was transferred to row B, mixed with a pipette, and repeated down the plate through row H. After mixing of samples in the well in row H, 100 μL is removed from the well in row H and discarded. This process generates a dilution series of the samples of 1:100 in row A to 1:1280 in row H. The plate was then incubated 1 hour at 37° C. Fluids were dumped from the plate and the plate was washed four times with 100 μL/well of PBS+0.05% tween 20. Goat anti-chicken IgY horseradish peroxidase conjugate diluted 1:1000 in Superblock was next added to each well (100 μL) and incubated 1 hour at 37° C. Plates were again washed four times with PBS-T. Next 100 uL of KPL SureBlue Reserve TMB Microwell Peroxidase Substrate (SeraCare 5120-0083) was added to each well and incubated 15 minutes at 37° C. The reaction was then stopped by the addition of 100 μL of 1.0 N HCl and the plate was read at 450 nm. Plots were constructed of optical density (O.D.) as a function of dilution for each sample. Maximal absorbance for clarified material from control non-immunized hens was ˜0.4. In contrast, antibodies purified from DWV-immunized hens could be diluted ˜10,000 before the absorbance dropped to this level. These results (FIG. 1) demonstrate that antibodies specific to VP1, VP2 and VP3 were generated. Also, the ELISA results show no appreciable difference in purified IgY concentration using a commercial IgY purification kit or the process using water dilution followed by freeze/thaw and hollow fiber concentration.

### Preparation of DWV Challenge

A bag containing 320 frozen honeybees from a commercial operation was crushed and suspended in 80 mL of PBS for a final volume of 88 mL. This solution was centrifuged at 5,000×g for 10 minutes. The insoluble lipid layer (top layer) and pellet were discarded, while the remaining supernatant was collected and transferred to a new centrifuge tube and centrifuged at 20,000×g for 20 minutes. Again, the lipid layer and pellet were discarded. The remaining supernatant was then centrifuged at 75,600×g (25,000 rpm) for 2 hours. The resulting pellet containing DWV was suspended in 1 mL PBS, while the supernatant was discarded. The temperature during all centrifugation steps should be held between 4-8° C. The DWV was then stored at (−80° C.) until use for larvae challenge.

### Larval Experiments

In order to test the effectiveness of the antibody, worker bee larvae were grown in vitro according to published protocols. A frame containing at least 25% eggs and 1^{st} instar larvae was selected from a hive and transported to the lab in an insulated box. The frame was kept humidified (i.e., draped in wet paper towels) during transport. One-day old A. mellifera larvae free of DWV are were grafted from the frame to queen cell cups (Mann Lake Ltd. Cat. #QC-520) inserted into each well 48-well tissue culture plates. Each cup contained 20 μL of Diet A at the time of grafting. Larvae were fed according to Schmehl's feeding schedule (see Tables 1-2) with two exceptions: 20 μL of Diet A being administered on day 2 rather than 20 μL of Diet B; and an additional 10 μL of Diet A containing treatment (antibody) and challenge (DWV) was given on day 0, post-grafting. Larvae receiving challenge were administered challenge only on day 0, while larvae receiving antibody received it at the desired concentration at each feeding. Feed directly was pipetted down the side of the cup to avoid drowning.

TABLE 1
Percent composition of larval feed. Antibody and
challenge were added at desired concentrations.

| | Royal Jelly | Glucose | Fructose | Yeast Extract | Water |
|---|---|---|---|---|---|
| Diet A | 44.25% | 5.30% | 5.30% | 0.90% | 44.25% |
| Diet C | 50.00% | 9.00% | 9.00% | 2.00% | 30.00% |

TABLE 2
Feeding schedule of larvae; days correspond to time since
grafting. Antibody groups were fed antibody in each feeding,
while challenge groups were only fed challenge on day 0.

| Day 0 | Day 1 | Day 2 | Day 3 | Day 4 | Day 5 |
|---|---|---|---|---|---|
| 20 μL | None | 20 μL | 30 μL | 40 μL | 50 μL |
| Diet A | | Diet A | Diet C | Diet C | Diet C |
| 10 μL | | | | | |
| Diet A with | | | | | |
| Treatment | | | | | |

The larvae were subjected to various treatment protocols shown in Table 3.

TABLE 3
Treatment groups

| Group | Weighted CT Value | TCID50 |
|---|---|---|
| Control (Feed Only) | N/A | 0 |
| 1:25 Aby | N/A | 0 |
| 1:100 Aby | N/A | 0 |
| 1:400 Aby | 37.2 | 0.0 |
| DWV | 25.3 | 4.9 |
| 1:25 Aby + DWV | 33.0 | 3.1 |
| 1:100 Aby + DWV | 29.2 | 4.0 |
| 1:400 Aby + DWV | 28.5 | 4.1 |

Larvae were then placed in a desiccator with a cup of supersaturated salt solution consisting of 80 g K₂SO₄, 200 g NaCl, and 500 mL H₂O and incubated at 35° C. in a humidified incubator. Mortality assessment and feeding occurred every 24 hours post-grafting. The negative control group received only feed. Three treatment groups received different antibody concentrations (3 dilutions, 1:25 dilution of concentrate, 1:100 dilution of concentrate, 1:400 dilution of concentrate), without challenge, to investigate the safety of feeding the antibodies to the larvae. The positive control group did not receive any feed, but was challenged. Three final treatment groups received feed spiked with anti-DWV antibodies produced in the eggs at different concentrations (3 dilutions, 1:25 dilution of concentrate, 1:100 dilution of concentrate, 1:400 dilution of concentrate) as noted in the table below. Larvae in these groups were simultaneous challenged with DWV variant A (10⁷ genome copies/larvae) by oral administration.

Larvae were monitored for 10 days post challenge and dead larvae were quantified. All dead larvae were removed daily, sorted by treatment group, and frozen for further analysis. Larvae from each treatment group were separated into “live” and “dead” pools. Larvae were considered “live” only if they survived until the experiment's endpoint.

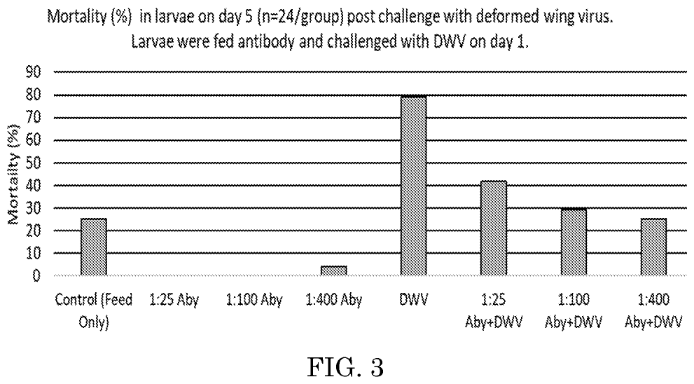

The minimum protective dose was determined statistically as the titer of antibody required to significantly improve DWV-associated mortality as compared to the no-antibody control. The results are shown in FIGS. 2-3, and Table 4.

TABLE 4

| | Cumulative % Mortality | | | | | | | | | |
|---|---|---|---|---|---|---|---|---|---|---|
| | Day | | | | | | | | | |
| | 1 | 2 | 3 | 4 | 5 | 6 | 7 | 8 | 9 | 10 |
| Control (Feed Only) | 0.0 | 4.2 | 4.2 | 12.5 | 25.0 | 25.0 | 25.0 | 25.0 | 25.0 | 25.0 |
| 1:25 Aby | 0.0 | 0.0 | 0.0 | 0.0 | 0.0 | 0.0 | 0.0 | 0.0 | 0.0 | 0.0 |
| 1:100 Aby | 0.0 | 0.0 | 0.0 | 0.0 | 0.0 | 0.0 | 0.0 | 0.0 | 0.0 | 0.0 |
| 1:400 Aby | 0.0 | 4.2 | 4.2 | 4.2 | 4.2 | 4.2 | 4.2 | 4.2 | 4.2 | 4.2 |
| DWV | 0.0 | 4.2 | 8.3 | 16.7 | 79.2 | 79.2 | 79.2 | 79.2 | 79.2 | 79.2 |
| 1:25 Aby + DWV | 0.0 | 0.0 | 8.3 | 8.3 | 41.7 | 41.7 | 41.7 | 41.7 | 41.7 | 41.7 |
| 1:100 Aby + DWV | 0.0 | 4.2 | 4.2 | 12.5 | 29.2 | 29.2 | 29.2 | 29.2 | 29.2 | 29.2 |
| 1:400 Aby + DWV | 0.0 | 0.0 | 0.0 | 4.2 | 25.0 | 25.0 | 25.0 | 25.0 | 25.0 | 25.0 |

### DWV RT-PCR

At the conclusion of each in vitro larvae experiment, larvae from each treatment group were tested for the presence of DWV using SYBR Green Real-Time PCR. Each pool of larvae was added to two volumes of PBS, crushed using a pipette tip, and vortexed for several seconds. 0.2 mL of each sample was added to a micro-centrifuge tube and spun at 2,000×g for 5 minutes. 100 μL of each resulting supernatant was added to 100 μL of PrepMan (ThermoFisher 4318930) and vortexed. These tubes were then placed on a heat block at 98° C. for 10 minutes. After heating, samples were micro-centrifuged at 20,627×g (15,000 rpm) for 10 minutes. The resulting supernatants were used as the template RNA samples for PCR. The following volumes (given per sample) were combined to create the master mix: 0.5 μL PAN DWV Forward primer, 0.5 μL PAN DWV Reverse primer, 12.5 μL 2× QuantiTect SYBR Green RT-PCR Master Mix, 8.75 μL nuclease-free water, 0.25 μL QuantiTect RT Mix, and 2.5 μL template RNA sample (total volume/well=25 μL).

### Results

As can be seen, the antibodies appeared generally safe, particularly at low levels. Further, administration of the antibodies significant improved survival. In contrast, by day 5, ˜80% of larvae in the control group that did not receive any antibody in their feed had died. The larvae receiving the antibody composition had a statically significant reduction in mortality, by at least 50% even at low levels. Higher dosages were associated with further improvements in survival (60-75% survival over untreated groups). Further, PCR analysis of the larvae showed a drop in viral titers in larval tissue in the treatment groups, as compared to the control group. The outcome of this research is surprising and exciting. Given that insects only have rudimentary immune systems and no adaptive immunity, the use of antibodies as a treatment protocol for preventing or treating DWV represents as significant advancement in the fight to reduce CCD and save honeybee populations.

1. A prophylactic and/or therapeutic food composition for control of deformed wing virus (DWV) in bees and/or bee larvae, said composition comprising anti-DWV antibodies dispersed in an edible base composition.

2. The food composition of claim 1, wherein said edible base composition is selected from the group consisting of a liquid solution, dry granules or powder, a compressed tablet, and a patty.

3. The food composition of claim 1, wherein said edible base composition comprises a carbohydrate source or is formulated for mixing with a carbohydrate source.

4. The food composition of claim 3, wherein said carbohydrate source is sucrose, corn syrup, sugar syrup, cane, or beet sugar.

5. The food composition of claim 1, wherein said composition further comprises one or more of amino acids, vitamins, fats/lipids, probiotics, enzymes/prebiotics, essential oils, yeast, plant polyphenols, phytonutrients, minerals, herbs, and proteins.

6. The food composition of claim 1, wherein said composition further comprises one or more antibiotics, miticides, formic acid, nutraceuticals, or combinations thereof.

7. The food composition of claim 1, wherein said anti-DWV antibodies comprise anti-DWV-A antibodies.

8. The food composition of claim 1, wherein said anti-DWV antibodies have been purified from chicken eggs.

9. The food composition of claim 8, wherein said chicken eggs are from hens immunized with recombinant VP1, VP2, VP3, and/or VP4 capsid proteins, or VLPs.

10. A method of prophylaxis or treatment of deformed wing virus in a bee colony, said method comprising: placing a composition according to claim 1 in a location where a bee or bee larvae from said colony will come into direct contact with said composition.

11. The method of claim 10, wherein said composition is placed inside a beehive.

12. The method of claim 10, wherein said composition is placed inside a brood box of a beehive.

13. The method of claim 10, wherein said composition is ingested by said bee or bee larvae at a therapeutically-effective dosage or over a therapeutically-effective time interval.

14. (canceled)

15. The method of claim 10, wherein said method is effective in prophylactically or therapeutically treating bee colony collapse disorder.

16. The method of claim 10, wherein bee colony is free of observable signs of deformed wing virus prior to said placing of said composition in a location where a bee or bee larvae from said colony will come into direct contact with said composition.

17. The method of claim 10, wherein said bee colony shows observable signs of deformed wing virus prior to said placing of said composition in a location where a bee or bee larvae from said colony will come into direct contact with said composition.

18. A method of preparing a prophylactic and/or therapeutic food composition for control of deformed wing virus (DWV) in bees and/or bee larvae, said method comprising:
generating recombinant VP1, VP2, VP3, and/or VP4 capsid proteins, or VLPs of DWV in an expression system;
immunizing hens with said recombinant VP1, VP2, VP3, and/or VP4 capsid proteins, or VLPs;
purifying anti-DWV antibodies from eggs laid by said immunized hens, and
combining said anti-DWV antibodies with an edible base composition for bees to yield said prophylactic and/or therapeutic food composition for control of deformed wing virus (DWV) in bees and/or bee larvae.

19. The method of claim 18, wherein said expression system is a baculovirus expression system.

20. The method of claim 19, wherein said VP1, VP2, VP3, and/or VP4 capsid proteins, or VLPs are codon-optimized for expression insect cells.

21. The method of claim 18, further comprising providing said composition in or near a beehive such that bees and/or larvae come into direct contact therewith for prophylaxis or treatment of deformed wing virus in the bees and/or larvae.

22. (canceled)


SEQUENCE LISTING (8 sequences)

SEQ ID NO: 1 - protein, 416 aa, Deformed wing virus
DNPSYQQSPRHFVPTGMHSLALGTNLVEPLHALRLDASGTTQHPVGCAPDEDMTVSSIASRYGLIRQVQWKKDHAKGSLLLQLDADPFVEQKIEGTNPISLYWFAPVGVVSSMFMQWRGSLEYRFDIIASQFHTGRLIVGYVPGLTASLQRQMDYMKLKSSSYVVFDLQESNSFTFEVPYVSYRPWWVRKYGGNYLPSSTDAPSTLFMYVQVPLIPMEAVSDTIDINVYVRGGSSFEVCVPVQPSLGLNWNTDFILRNDEEYRAKNGYAPYYAGVWHSFNNSNSLVFRWGSASDQIAQWPTITVPRGELAFLRIRDAKQAAVGTQPWRTMVVWPSGHGYNIGIPTYNAERARQLAQHLYGGGSLTDEKAKQLFVPANQQGPGKVSNGNPVWEVMRAPLATQQAHIQDFEFVEAVPE

SEQ ID NO: 2 - protein, 253 aa, Deformed wing virus
MDNPNPGPDGEGEVELEKDSNVVLTTQRDPSTSIPAPTSVKWSRWTSNDVVDDYATITSRWYQIAEFVWSKDDPFDKELARLILPRALLSSIEANSDAICDVPNTIPFKVHAYWRGDMEVRVQINSNKFQVGQLQATWYYSDHENLNIQTKRSVYGFSHMDHALISASASNEAKLVIPFKHVYPFLPTRVVPDWTTGILDMGTLNIRVIAPLRMSATGPTTCNVVVFIKLNNSEFTGTSSGKFYANQIRAKPE

SEQ ID NO: 3 - protein, 258 aa, Deformed wing virus
GEESRNTTVLDTTTTLQSSGFGRAFFGEAFNDLKTLMRRYQLYGQLLLSVTTDKDIDHCMFTFPCLPQGLALDIGSAGSPHEIFNRCRDGIIPLIASGYRFYRGDLRFKIVFPSNVNSNIWVQHRPDRRLKGWSEAKIVNCDAVSTGQGVYNHGYASHIQITRVNNVIELEVPFYNATCYNYLQAFNPSSAASSYAVSLGEISVGFQATSDDIAAIVNKPVTIYYSIGDGMQFSQWVGYQPMMILDQLPAPVVRAVPE

SEQ ID NO: 4 - protein, 21 aa, Deformed wing virus
MDRVLNLAEGLLNNTVGGCNM

SEQ ID NO: 5 - protein, 38 aa, Artificial Sequence
  Synthetic peptide GP67 signal sequence
MLLVNQSHQGFNKEHTSKMVSAIVLYVLLAAAAHSAFA

SEQ ID NO: 6 - DNA, 1407 bp, Artificial Sequence
  Recombinant expression vector
gaattcgccgccaccatgctgctggtcaaccagtctcaccagggtttcaacaaagaacacacctccaagatggtgtccgccatcgtgctgtacgtgctgttggctgctgctgctcactccgctttcgctgacaacccttcctaccagcagtcccctcgtcacttcgtgcctaccggaatgcactctctggctctgggtactaacctggtcgagcctctgcacgctctgcgtttggacgcttccggtactactcagcaccctgtgggttgcgctccagacgaggacatgaccgtgtcctctatcgcttcccgttacggcctgatccgtcaggtgcagtggaagaaggaccacgctaagggttccctgctgttgcagttggacgctgaccctttcgtcgagcagaagatcgagggcactaaccccatcagcctgtattggttcgctcccgtgggtgtcgtgtcctccatgttcatgcaatggcgcggttccctggaataccgcttcgacatcattgcttcccagttccacaccggtcgtctgatcgtgggttacgtgccaggactgaccgcttccttgcagcgtcagatggactatatgaagctgaagtcctcctcctacgtggtgttcgacctgcaagagtccaactccttcaccttcgaggtgccctacgtgtcctaccgtccttggtgggttcgcaaatacggtggcaactacctgccttcctctaccgacgctccctccactctgtttatgtacgtgcaggtcccactgatccccatggaagctgtgtccgacaccatcgacatcaacgtgtacgtgcgtggtggttccagcttcgaagtgtgcgtgccagtgcagccttctctgggcctgaactggaacaccgacttcatcctgcgtaacgacgaagagtaccgtgctaagaacggttacgctccctactacgctggtgtctggcactccttcaacaactccaactctctggtgttccgttggggttccgcttccgaccagatcgctcagtggcctaccatcactgtgcctaggggagagctggctttcctgcgtatccgtgacgctaagcaggctgctgtgggtactcagccttggcgtactatggtcgtgtggccttccggtcacggttacaacatcggtatccccacctacaacgctgagagggctcgtcagctggctcagcacttgtacggtggtggatctctgaccgacgagaaggctaagcagctgttcgtgcccgctaaccagcaaggtcctggcaaggtgtccaacggcaaccctgtgtgggaagtgatgagggctcctctggctacccagcaggctcacatccaggacttcgagttcgtcgaggctgtgcccgaacatcaccaccaccatcattaatgaaagctt

SEQ ID NO: 7 - DNA, 918 bp, Artificial Sequence
  Recombinant expression vector
gaattcgccgccaccatgctgctggtcaaccagtctcaccagggtttcaacaaagaacacacctccaagatggtgtccgccatcgtgctgtacgtgctgttggctgctgctgctcactccgctttcgctatggacaaccccaatcctggtcctgacggcgagggcgaagtggaactggaaaaggactccaacgtggtgctgaccactcagcgtgacccctccacttctatccctgctcctacctccgtgaagtggtcccgttggacctctaacgacgtggtggacgactacgctaccatcacctccaggtggtatcagatcgctgagttcgtgtggtccaaggacgaccccttcgacaaagagctggctcgtctgatcctgcctcgtgctctgctgtcctccatcgaggctaactccgacgctatctgcgacgtgcccaacactatccccttcaaggtgcacgcttactggcgtggcgacatggaagtgcgtgtgcagatcaactccaacaagttccaagtgggccagctccaggctacctggtactactctgaccacgagaacctgaacatccagaccaagcgttccgtgtacggtttctcccacatggaccacgctctgatctccgcttccgcttccaacgaagctaagctggtcatccctttcaagcacgtgtaccctttcttgcccactcgtgtggtgcctgactggaccactggtatcctggacatgggtactctgaatatccgtgtgatcgctcccctgcgtatgtccgctaccggtcctactacttgcaacgtggtcgtgttcatcaagctgaacaactccgagttcaccggcacctcttccggcaagttctacgctaaccagatccgtgctaagcccgagcaccaccaccatcaccactaatgaaagctt

SEQ ID NO: 8 - DNA, 933 bp, Artificial Sequence
  Recombinant expression vector
gaattcgccgccaccatgctgctggtcaaccagtctcaccagggtttcaacaaagaacacacctccaagatggtgtccgccatcgtgctgtacgtgctgttggctgctgctgctcactctgctttcgctggcgaggaatcccgtaacaccaccgtgctggacaccaccaccactctgcaatcctccggtttcggtcgtgctttcttcggcgaggctttcaacgacctcaagaccctgatgcgccgttaccagctgtacggtcaactgctgctgtccgtgaccaccgacaaggacatcgaccactgcatgttcactttcccatgcctgcctcaaggcctggctctggacattggttctgctggttctccccacgagatcttcaaccgttgccgtgacggtatcatccctctgatcgcttccggttaccgtttctaccgtggcgacctgcgtttcaagatcgtgttcccctccaacgtgaactccaacatctgggtgcagcaccgtcctgaccgtcgtttgaaaggctggagcgaggctaagatcgtgaactgcgacgctgtgtccaccggacagggcgtttacaaccacggttacgcttcccacatccagatcacccgtgtgaacaacgtgatcgagctggaagtgcccttctacaacgctacctgctacaactacctccaggcgttcaacccctcctccgctgcttcttcctacgctgtctctctgggcgagatctccgtgggattccaggctacctccgacgatatcgctgctatcgtgaacaagcccgtgaccatctactactccatcggcgacggcatgcagttctcccagtgggtcggataccagcctatgatgatcctggaccagctgcctgctcctgtcgtgcgtgctgttcctgaacaccaccatcaccaccactaatgaaagctt